(19) United States
(12) Patent Application Publication
(10) Pub. No.: US 2018/0305742 A1
(43) Pub. Date: Oct. 25, 2018

(54) USE OF ESTROGENIC COMPOUNDS TO MANIPULATE THE BACTERIAL COMPOSITION OF VAGINAL COMMUNITIES

(71) Applicant: University of Idaho, Moscow, ID (US)

(72) Inventors: Larry J. Forney, Troy, ID (US); Karol S. Gliniewicz, Moscow, ID (US)

(73) Assignee: University of Idaho, Moscow, ID (US)

(21) Appl. No.: 15/957,686

(22) Filed: Apr. 19, 2018

Related U.S. Application Data
(60) Provisional application No. 62/488,380, filed on Apr. 21, 2017.

(51) Int. Cl. C12Q 1/689

(52) U.S. Cl. CPC C12Q 1/689; C12Q 2600/16; C12Q 2600/106

(57) ABSTRACT

Disclosed herein are embodiments of a method for using estrogenic compounds to manipulate the bacterial composition of vaginal communities of female subjects. The method may include titrating a dose of an estrogenic compound to provide a female subject with a personalized effective dose, such as a minimum effective dose, sufficient to attain and/or maintain one or more determined target values of a relative abundance of vaginal Lactobacillus in the vaginal microbiota, an absolute abundance of vaginal Lactobacillus in the vaginal microbiota, a vaginal pH, a vaginal lactic acid concentration, or any combination thereof.

### CROSS-REFERENCE TO RELATED APPLICATION

This application claims the benefit of the earlier filing date of Provisional Application No. 62/488,380, filed Apr. 21, 2017, which is incorporated by reference herein in its entirety.

### FIELD

This invention concerns embodiments of a method for using estrogenic compounds to manipulate the bacterial composition of vaginal microbiota.

### BACKGROUND

The vaginal microbiota is home to diverse bacterial communities that play an important role in maintaining health and protecting individuals from infectious disease. In most reproductive-age women, these communities are dominated by species of Lactobacillus and their presence is a hallmark of health. Their protective ability is attributed to the production of lactic acid and other antimicrobial substances. Lactic acid is responsible for reducing the pH of the vaginal environment, which is thought to make it inhospitable to other invading organisms and precluding the growth of unwanted organisms. However, roughly one quarter of all reproductive-age women have communities not dominated by lactobacilli. Longitudinal studies have shown that this state can be transient lasting for just a few days, while in other instances, it persists for many weeks.

Vaginal dysbiosis is a microbial imbalance where normally predominant species are diminished in abundance and less predominant species become more abundant and/or predominant. Vaginal dysbiosis may be asymptomatic or may lead to symptoms such as malodor, burning, itching, discharge, inflammation, dyspareunia, or combinations thereof. Epidemiological studies suggest that women with vaginal dysbiosis may be at higher risk for various maladies that include a higher risk of sexually-transmitted infections, including HIV. Vaginal dysbiosis also can reduce efficacy of pre-exposure prophylaxis regimens intended to reduce a woman's risk of acquiring a sexually-transmitted infection.

### SUMMARY

Embodiments of a method for using estrogenic compounds to manipulate the bacterial composition of vaginal microbiota are disclosed. Certain disclosed embodiments of the method include: (i) obtaining a vaginal sample from a female subject, the vaginal sample comprising vaginal microbiota; (ii) obtaining initial analysis results of the vaginal sample, the initial analysis results comprising a relative abundance of Lactobacillus in the vaginal microbiota, an absolute abundance of Lactobacillus in the vaginal microbiota, a vaginal pH, a vaginal lactic acid concentration, or any combination thereof; (iii) determining, based at least in part on the initial analysis results, an effective dose of an estrogenic compound for administration to the female subject to shift the relative abundance of vaginal Lactobacillus in the vaginal microbiota, the absolute abundance of vaginal Lactobacillus in the vaginal microbiota, the vaginal pH, the vaginal lactic acid concentration, or any combination thereof toward one or more determined target values; and (iv) administering one or more effective doses of the estrogenic compound to the female subject over an effective period of time, in response to which the relative abundance of vaginal Lactobacillus in the vaginal microbiota, the absolute abundance of vaginal Lactobacillus in the vaginal microbiota, the vaginal pH, the vaginal lactic acid concentration, or any combination thereof shifts toward or attains the one or more determined target values.

Determining, based at least in part on the initial analysis results, the effective dose of the estrogenic compound for administration to the female subject may include comparing one or more of (i) the relative abundance of Lactobacillus in the vaginal microbiota to a determined relative abundance of Lactobacillus target value, (ii) the absolute abundance of Lactobacillus in the vaginal microbiota to a determined absolute abundance of Lactobacillus target value, (iii) the vaginal pH to a determined vaginal pH target value, or (iv) the vaginal lactic acid concentration to a determined vaginal lactic acid concentration target value to provide a comparison. An effective dose of the estrogenic compound is then determined based at least in part on the comparison. In some embodiments, an effective dose is administered daily or weekly to the female subject.

In any or all of the above embodiments, the estrogenic compound may be estradiol, estrone, estriol, ethinyl estradiol, estrone sulfate, equilin, equilin sulfate, equilenin, estradiol 17 beta-cypionate, estradiol valerate, estradiol acetate, estradiol undecylate, polyestradiol phosphate, ethinylestradiol, methylestradiol, mestranol, moxestrol, quinestrol, benzestrol, dienestrol, dienestrol acetate, disethylstilbestrol dipropionate, fosfestrol, hexestrol, methestrol dipropionate, chlorotrianisene, doisynoestrol, methallenestril, 27-hydroxycholesterol, dehydroepiandrosterone (DHEA), 7-oxo-DHEA, 7α-hydroxy-DHEA, 16α-hydroxy-DHEA, 7β-hydroxepiandrosterone, 4-androstenedione, 5-androstenediol, 3α-androstanediol, a phytoestrogen, a mycoestrogen, or any combination thereof.

In any or all of the above embodiments, the initial analysis results may further comprise a relative abundance of one or more particular Lactobacillus species in the vaginal sample, an absolute abundance of one or more particular Lactobacillus species in the vaginal sample, ratios of two or more particular Lactobacillus species in the vaginal sample, or any combination thereof. By way of example, the particular Lactobacillus species may include L. crispatus, L. jensenii, L. gasseri, L. iners, L. coleohominis, L. johnsonii, or any combination thereof.

In any or all of the above embodiments, the method may further include (v) obtaining a subsequent vaginal sample from the female subject a period of time after beginning administration of the one or more effective doses of the estrogenic compound, the subsequent vaginal sample comprising a subsequent vaginal microbiota; (vi) obtaining subsequent analysis results of the subsequent vaginal sample, the subsequent analysis results comprising a relative abundance of Lactobacillus in the subsequent vaginal microbiota, an absolute abundance of Lactobacillus in the subsequent vaginal microbiota, a subsequent vaginal pH, a subsequent vaginal lactic acid concentration, or any combination thereof; (vii) adjusting, based at least in part on the subsequent analysis results, the effective dose of the estrogenic compound to provide an adjusted effective dose of the estrogenic compound for administration to the female subject; and (viii) administering one or more adjusted effective doses of the estrogenic compound to the female subject over an effective period of time, in response to which the one or more determined target values of the relative abundance of vaginal Lactobacillus in the vaginal microbiota, the absolute abundance of vaginal Lactobacillus in the vaginal microbiota, the vaginal pH, the vaginal lactic acid concentration, or any combination thereof is attained and/or maintained. The effective dose of the estrogenic compound may be titrated by performing the steps of obtaining a subsequent vaginal sample from the female subject, obtaining subsequent analysis results of the subsequent vaginal sample, and adjusting the effective dose of the estrogenic compound periodically, such as once every week or once every two weeks for an effective period of time to obtained a desired result, such as a period of 4-16 weeks after beginning administration of the one or more effective doses of the estrogenic compound. In some embodiments, the effective dose is further titrated by performing the steps of obtaining a subsequent vaginal sample from the female subject, obtaining subsequent analysis results of the subsequent vaginal sample, and adjusting the effective dose of the estrogenic compound once every 3-12 months after the period of 4-16 weeks.

In any or all of the above embodiments, the effective dose may be a minimum dose of the estrogenic compound effective to provide the female subject with a vaginal microbiota dominated by Lactobacillus species. The method further includes administering the minimum dose of the estrogenic compound to the female subject, thereby providing the female subject with a vaginal microbiota dominated by Lactobacillus species. In any or all of the above embodiments, the effective dose may be a daily dose within a range of from 0.05 μg to 2 mg of the estrogenic compound. In any or all of the above embodiments, the effective dose may be administered in any suitable manner including, without limitation, orally, vaginally, or transdermally. Administering the effective does may include administering an amount of a pharmaceutical composition comprising the effective dose of the estrogenic compound and a pharmaceutically acceptable carrier. In some embodiments, the pharmaceutical composition is provided as an oral dosage form, a vaginal ring, a transdermal patch, or a topical cream, gel, ointment, paste, or spray comprising the pharmaceutical composition.

In any or all of the above embodiments, the female subject may be experiencing one or more vaginal symptoms of malodor, burning, itching, discharge, inflammation, or dyspareunia, and administering the effective dose of the estrogenic compound to the female subject mitigates at least one of the one or more vaginal symptoms. In any or all of the above embodiments, the female subject may be sexually active, and administering the effective dose of the estrogenic compound to the female subject reduces the female subject's risk of acquiring a sexually transmitted infection (STI) compared to a risk of acquiring an STI in a female subject in the absence of estrogenic compound administration. In any or all of the above embodiments, the female subject may be a woman of reproductive age taking an antiviral drug in a pre-exposure prophylaxis (PrEP) regimen or selected to take an antiviral drug in a PrEP regimen, and administering the effective dose of the estrogenic compound to the female subject increases efficacy of the PrEP regimen compared to an efficacy of a PrEP regimen for a female subject taking the antiviral drug in the absence of estrogenic compound administration.

The foregoing and other objects, features, and advantages of the invention will become more apparent from the following detailed description, which proceeds with reference to the accompanying figures.

### SEQUENCE LISTING

The nucleic acid sequences listed in the accompanying sequence listing are shown using standard letter abbreviations for nucleotide bases, as defined in 37 C.F.R. § 1.822. Only one strand of each nucleic acid sequence is shown, but the complementary strand is understood as included by any reference to the displayed strand. The Sequence Listing is submitted as an ASCII text file, created on Apr. 17, 2018, 4.29 kB, which is incorporated by reference herein. In the accompanying sequence listing:

SEQ ID NOs: 1-14 are oligonucleotide primers for amplifying the 16S rRNA gene, where the underlined sequences are the universal 16S rRNA primers 27F and 534R, which include seven different 27F primer sequences to capture a broad spectrum of taxa. The bold letters denote universal sequence tags CS1 and CS2, and the italicized bases are added to the template specific primers to introduce variability of base calls during sequencing.

SEQ ID NOs: 15 and 16 are oligonucleotide adapter primers that include the specific sequences P5 as well as P7 for dual indexing, and an 8-bp barcode is denoted by eight italicized Ns, which allow simultaneous sequencing using relatively few barcoded adapter primers. The bold letters denote universal sequence tags CS1 and CS2

SEQ ID Nos: 17-20 are universal sequence tags CS1, CS2, CS1rc, and CS2rc.

### DETAILED DESCRIPTION

Embodiments of a method for the use of estrogenic compounds to manipulate the bacterial composition of vaginal microbiota are disclosed.

### I. Definitions

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

Unless otherwise indicated, all numbers expressing quantities of components, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximates unless the word “about” is recited.

Definitions of common terms in chemistry may be found in Richard J. Lewis, Sr. (ed.), Hawley's Condensed Chemical Dictionary, published by John Wiley & Sons, Inc., 1997 (ISBN 0-471-29205-2). Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar references.

In order to facilitate review of the various embodiments of the disclosure, the following explanations of specific terms are provided:

Bacterial vaginosis (BV): A vaginal condition caused by the overgrowth or over-abundance of certain species of bacteria naturally found in the vagina, which may result in depopulation of lactobacilli. The cause(s) that trigger the depopulation of lactobacilli, changes in microbial community structure, and the overgrowth of other organisms are not fully understood. However, an increased incidence of BV is known to be positively correlated with various behaviors, including multiple sex partners, the frequency of intercourse, and douching (Simpson et al., J. Pediatr. Adolesc. Gynecol. 17:249-255 (2004)). Since the development of BV has not been attributed to the presence or absence of any single bacterial taxon, it is commonly diagnosed based on the existence of three of the following four symptoms: (a) thin, homogeneous, malodorous discharge; (b) vaginal fluid pH>4.5; (c) an amine odor from vaginal fluid when 10% KOH is added; and (d) the presence of “clue” cells (vaginal epithelial cells with adherent bacteria that obscure cell margins) (Amsel et al., Am. J. Med. 74:14-22 (1983)). Alternatively, the abundance of clue cells in Gram-stained vaginal smears can also be used as a means to diagnose BV (Nugent et al., J. Clin. Microbiol. 29:297-301 (1991)).

Dysbiosis: A microbial imbalance where normally predominant species are diminished in abundance and less predominant species become more abundant and/or predominant. Vaginal dysbiosis is a microbial imbalance in the vagina.

Effective dose or therapeutic dose: An amount sufficient to provide a beneficial, or therapeutic, effect to a subject or a given percentage of subjects.

Estrogen: The primary female sex hormone. The three primary naturally occurring estrogens in humans include estrone, estradiol, and estriol (structures below). A fourth estrogen, estetrol, is produced during pregnancy. Estradiol is the major endogenous estrogen produced by ovaries and is capable of the fullest range of estrogen effects because it is received by estrogen receptors. Estradiol can also be produced by conversion from a number of precursors in the adrenal glands. Estrone is typically produced by special fat cells, and is the major estrogenic form found in naturally-menopausal women who are not taking hormone replacement therapy (HRT). It is not directly active in as many tissues as estradiol, but can be readily converted to estradiol for actual use. Estriol is a metabolic waste product of estradiol metabolism that can still have some effects upon a limited number of estrogen receptors. Estriol is formed in the liver and is 8% as potent as estradiol and 14% as potent as estrone. Once estriol is bound to an estrogen receptor, it blocks the stronger estradiol. Thus it is considered to have both estrogenic and anti-estrogenic actions. Ethinyl estradiol is a derivative of estradiol. Ethinyl estradiol is an orally active estrogen used in almost all formulations of combined oral contraceptives (COCs), being nearly the exclusive estrogen used for this purpose.

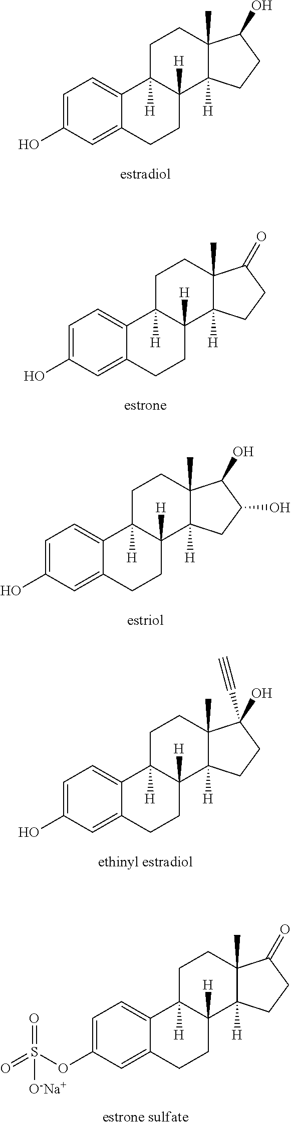

Estrogenic: Having the properties of, or similar to, an estrogen; having the effects of, or similar to, an estrogen.

Estrogenic compounds may be natural (e.g., mycoestrogens, phytoestrogens) or synthetic. Synthetic estrogenic compounds include, but are not limited to, estrogen esters (e.g., estradiol valerate, estradiol cypionate (such as estradiol 17 beta-cypionate), estradiol acetate, estradiol undecylate, polyestradiol phosphate, estradiol benzoate), ethinylestradiol, methylestradiol, mestranol, moxestrol, quinestrol, stilbestrols (benzestrol, dienestrol, dienestrol acetate, disethylstilbestrol dipropionate, fosfestrol, hexestrol, methestrol dipropionate), chlorotrianisene, doisynoestrol, and methallenestril, among others. Other naturally-occurring estrogenic compounds include equine-derived estrogenic compounds such as equilin, equilin sulfate (structure below), equilenin, and estrone sulfate. Some estrogen metabolites are also estrogenic compounds, e.g., 27-hydroxycholesterol, dehydroepiandrosterone (DHEA), 7-oxo-DHEA, 7α-hydroxy-DHEA, 16α-hydroxy-DHEA, 7β-hydroxepiandrosterone, 4-androstenedione, 5-androstenediol, and 3α-androstanediol.

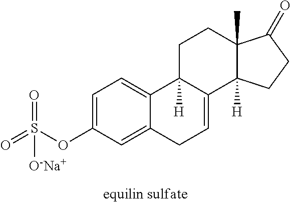

Microbiota: Microorganisms localized to a distinct environment. For example, “vaginal microbiota” are an assemblage of one or more species of microorganisms that are localized to, or found in, a vagina. Microorganisms include bacteria (Archaea, Eubacteria), yeast, fungi, protozoa. For certain embodiments, vaginal “microorganisms” shall be understood to include viruses. “Normal vaginal microbiota” are a population of microorganisms that localize to the vagina in a normal, that is, a non-pathological or non-pathogenic, state. For example, a sample of normal vaginal microbiota is obtained from a woman without a vaginal pathology, that is, from a woman with no sign or symptom corresponding to or resulting from a pathology of the vagina. Normal vaginal microbiota can be obtained from a woman with a pathology of an organ or tissue other than the vagina. In a medical context, the term “microflora” is often used synonymously with the term “microbiota.”

Molecular indicator of identity: Any molecule that differs between species or strains, and for which the difference can be detected. Most typically, a molecular indicator of identity is polymorphic nucleic acid, or a polymorphic polypeptide encoded by a polymorphic nucleic acid. The term “polymorphic” or “polymorphism” refers to a nucleic acid or polypeptide that exists in two or more variant forms. The variant forms may be detectable at the molecular level (e.g., at the nucleic acid or polypeptide level) or may be detectable as functional variants, for example, by phenotypic differences between species or strains. In some cases, a molecular indicator of identity is not directly encoded by a polymorphic polynucleotide. For example, polymorphic glycoproteins can be detected based on differences in their carbohydrate moieties. In addition, in some cases the molecular indicator of identity can be a metabolic product that differs between species, for example a detectable metabolite, such as a secondary metabolite, that differs between species.

Mycoestrogen: An estrogenic compound that is a mold metabolite of Fusarium species. Exemplary mycoestrogens include zearalenone, zearalenol, and zearalanol.

Pharmaceutically acceptable carrier: The pharmaceutically acceptable carriers (vehicles) useful in this disclosure are conventional. Remington: The Science and Practice of Pharmacy, The University of the Sciences in Philadelphia, Editor, Lippincott, Williams, & Wilkins, Philadelphia, Pa., 21^{st} Edition (2005), describes compositions and formulations suitable for pharmaceutical delivery of one or more therapeutic compositions and additional pharmaceutical agents. In general, the nature of the carrier will depend on the particular mode of administration being employed. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. In some examples, the pharmaceutically acceptable carrier is a non-naturally occurring or synthetic carrier. The carrier also can be formulated in a unit-dosage form that carries a preselected therapeutic dosage of the active agent, for example in a pill.

Phytoestrogen: An estrogenic compound derived from a plant. Phytoestrogens include a phenolic ring that binds to estrogen receptor and have a molecular weight similar to estrogens. Phytoestrogens include polyphenols (e.g., resveratrol), flavonoids (e.g., eriodictyol, hesperetin, homoeriodictyol, naringenin, 8-prenylnaringenin, apigenin, luteolin, tangeritin, fisetin, kaempferol, myricetin, pachypodol, quercetin, rhamnazin, proanthocyanides), isoflavonoids (e.g., daidzein, formonenetin, genistein, biochanin A, clycitein, equol), coumestans (e.g., coumestrol, 4′-methoxycoumestrol, repensol, trifoliol).

Polymorphic nucleic acid: A nucleic acid characterized by polymorphic polynucleotide sequences, that is, polynucleotide sequences with one or more nucleotide differences when aligned across a window of comparison. Such differences can be detected by determining the nucleotide sequence of the polymorphic polynucleotide, that is, by sequencing the polynucleotide, or at least a portion thereof, using any known methods, including automated methods, for sequencing nucleic acids. Alternatively, a polymorphism in a nucleic acid can be detected by a variety of techniques including RFLP, AFLP, SSCP, SNP, etc. A polymorphic nucleic acid can include a “phylogenetically informative gene,” that is, a functional genetic element that differs between species. A phylogenetically informative gene is one in which the differences in nucleotide sequence reflect the evolutionary relationships of organisms. An “rRNA gene” is one exemplary polymorphic nucleic acid. The rRNA genes encode the ribonucleic acid (“RNA”) components of ribosomes, and can be categorized based on the size of the ribosomal component in which the encoded RNA is localized. Prokaryotic rRNA genes include: the 16S rRNA gene, the 23S rRNA gene and the 5S rRNA gene. Eukaryotic rRNA genes include the 18S, 28S and 5.8S rRNA genes, respectively.

Predominant species: A predominant species may be the most numerically frequent species in a mixed sample or population, or a predominant species may be one of several numerically frequent species present in a sample or population comprising multiple species. In some embodiments, a predominant species is at least 10% of the sample or population. For example, a predominant species can be at least 20%, or at least 30%, frequently greater than about 40%, or greater than 50% of the community. In some cases, the predominant species is often than about 60%, sometimes greater than about 70%, and can be greater than 80% or even 90% of the sample or community. In another embodiment, a predominant species is at least 2× as abundant in the sample as another species of microorganism. Alternatively, the predominant species is at least 3× as abundant in the sample as other organisms. In some cases, the predominant species is at least 4×, or at least about 5×, or even as much as 10× as abundant in the sample or population than another species of microorganism.

Sexually transmitted infection/disease (STI, STD): An infection transmitted by vaginal, oral, or anal sexual intercourse. STIs include, but are not limited to, chlamydia, chancroid, crabs (pubic lice), genital herpes, genital warts, Hepatitis B, human immunodeficiency virus/acquired immunodeficiency syndrome, human papilloma virus, trichomoniasis, molluscum contagiosum, pelvic inflammatory disease, syphilis, gonorrhea, and yeast infections.

Subject: The term “subject” refers to an animal or human subjected to a treatment, observation or experiment. In certain disclosed embodiments, the subject is a human.

Therapeutic time window: The length of time during which an effective, or therapeutic dose, of a compound remains therapeutically effective in vivo.

Titrate: As used herein, the term “titrate” refers to adjusting the dose of an administered therapeutic agent (e.g., an estrogenic compound) to achieve a desired result.

### II. Vaginal Microbiota and Dysbiosis

Most commonly, the predominant species of the vaginal microbiota is a species of bacteria, or a combination of species of bacteria. Nonetheless, the predominant species of microbiota can also include species of yeast, species of fungi and species of viruses. However, normal vaginal microbiota varies among women, and varies statistically between women of different racial and/or ethnic backgrounds. Dysbiosis occurs when there is a disruption or imbalance in the normal vaginal microbiota such as when the normally predominant species are diminished in abundance and less abundant species become more abundant and/or predominant. Dysbiosis also may be accompanied by decreases in lactic acid and/or an increase in vaginal pH.

In some women, the predominant species of microbiota are selected from among Lactobacillus crispatus, Lactobacillus iners, Lactobacillus jensenii, Lactobacillus gasseri, Lactobacillus coleohominis, Lactobacillus johnsonii, Staphylococcus sp., Streptococcus sp., Atopobium vaginae, Lachnospiraceae sp., Megasphaera sp., Enterococcus faecalis, Peptoniphilus sp., Anaerococcus sp., Micromonas sp., Gemella palaticanis, Dialister sp., Clostridaceae sp. e.g., Clostridium perfringens, Aerococcus sp., Veillonella sp., Finegoldia magna, Granulicatella elegans, Gardinerella vaginalis, Pseudomonas sp., Mycoplasma sp., Mobiluncus muleiri, Peptostreptococcus anaerobis, Escherichia coli, Shigella sp., or a bacterium of the order Clostridiales. In many women, the predominant species is one or more of Lactobacillus crispatus, Lactobacillus iners, Lactobacillus jensenii, or Lactobacillus gasseri.

In some studies, Lactobacillus species have been found to be dominant in more than 70% of women overall, although there are variations amongst ethnic groups. A large cross-sectional study of 396 women in four ethnic groups showed that there are five kinds of communities found amongst all the women sampled. These are referred to as Community State Types (CST), which differ in terms of the kinds and abundances of bacterial taxa present. Four of these CST have one of four Lactobacillus species as a dominant member. The four species found are L. crispatus, L. jensenii, L. gasseri, and L. iners. The fifth (called CST IV), is not dominated by any species of Lactobacillus. While all five CST are found in all four ethnic groups, the proportions of each CST differ among ethnic groups. Ravel et al. showed that Lactobacillus species were dominant in 80.2% and 89.7% of Asian and white women, but this was the case in only 59.6% and 61.9% of black and Hispanic women (PNAS 2011, 108:4680-4687). Overall, 27% of the women sampled had vaginal communities in which Lactobacilli were not dominant.

Longitudinal studies have shown that Lactobacillus-depleted communities can be transient lasting just a few days, while in other instances they persist for many weeks. Some women with Lactobacillus-depleted communities remain asymptomatic and healthy. However, such women may be at higher risk for various maladies such as sexually transmitted infections.

A principle factor governing the composition of the vaginal microbial community is a woman's estrogen level. Each woman may have a threshold estrogen level above which her vaginal community is dominated by one or more species of Lactobacillus. As long as the woman's estrogen level is above that threshold, the vaginal community may remain dominated by the one or more species of Lactobacillus although the relative abundance of total Lactobacillus species and/or the individual amounts of each Lactobacillus species may fluctuate over time. However, if the woman's estrogen level drops below the threshold, her vaginal community may become Lactobacillus depleted. The threshold estrogen level varies from woman to woman. A woman's estrogen level changes during puberty, pregnancy, and menopause, and also fluctuates throughout the menstrual cycle. Additionally, the efficacy of the circulating estrogen level varies depending on genetic variations in estrogen receptors. The correlation between secreted estrogen and circulating estrogen levels also varies among women and the circulating estrogen level may be at least partially influenced by the gut microbiota.

The pH of the vagina is thought to be another principle factor in governing the composition of the vaginal microbial community in reproductive age women. A normal vaginal pH is somewhat acidic, typically within a range of pH 3.5-5.5, such as within a range of pH 3.5-4.5. A low pH environment selects for various acid-tolerant bacterial populations that can colonize and reproduce under such conditions, while precluding those that cannot (Pybus & Onderdonk Microbes Infect. 1:285-292 (1999)). Shifts in the structure of the vaginal microbial community that result in replacement of lactobacilli as the numerically dominant species, regardless of the cause, are associated with an increased pH. At increased pH, abnormal flora such as yeasts and various anaerobes and bacterial species associated with BV can proliferate. It is not clear, however, whether the pH shift is a cause or a consequence of differences in community composition. It seems that the production of lactic acid per se is important, but the particular species of Lactobacillus present is less so since it varies among women.

Bacterial vaginosis (BV) is the most frequently cited cause of vaginal discharge or malodor, and the most common vaginal disorder of reproductive age women. Symptoms of BV can include malodor, burning, itching, discharge, inflammation, dyspareunia, or combinations thereof. However, in some women, BV is asymptomatic. BV is associated with serious adverse sequelae including infertility, endometritis, and pelvic inflammatory disease, as well as an increased risk of human immunodeficiency virus, chlamydia (caused by Chlamydia trachomatis), gonorrhea (caused by Neisseria gonorrhoeae), and other sexually-transmitted infections. During pregnancy, BV is associated with several adverse outcomes including preterm delivery of low birthweight infants, spontaneous abortion, premature rupture of membranes, amniotic fluid infections, postpartum endometritis, and endometritis following Cesarean section. The prevalence of BV among women varies widely and depends on the subject population. BV has been found to be present in 10% to 20% of white, non-Hispanic women and 30% to 50% of African-American women.

One strategy to reduce the risk of HIV infection is to prophylactically administer antiviral drugs, such as tenofovir. However, it has been found that vaginal dysbiosis can reduce efficacy of pre-exposure prophylaxis regimens (PrEP) intended to reduce a woman's risk of acquiring a sexually-transmitted infection, such as HIV. It has been shown that “the vaginal microbiome doesn't just influence infection risk, it can also directly interfere with PrEP. In women whose microbiome contained less than 50% lactobacilli, tenofovir gel protected only 18% of the women who received it. The efficacy jumped to 61% when the proportion of Lactobacillus species was above 50%.” (http://www.sciencemag.org/news/2016/07/vaginal-bacteria-species-can-raise-hiv-infection-risk-and-undermine-prevention).

Vaginal communities undergo significant changes at various stages in a woman's lifespan that are directly linked to the level of estrogen in the body. There is mounting evidence for direct causal relationship, wherein estrogen exerts control over the kinds and amounts of resources that are available to vaginal microbiota, which in turn shapes a species composition of these communities. Changes in the relative abundance of vaginal lactobacilli are associated with estrogen levels during a woman's lifespan. These changes are seen during puberty, pregnancy, and menopause. During puberty and pregnancy, rising levels of estrogen are accompanied by an increased abundance of lactobacilli. During menopause the opposite occurs with decreasing levels of estrogen being accompanied by decreased numbers of lactobacilli. Atrophic vaginitis develops in 25-50% of postmenopausal women and may be characterized by symptoms of vaginal itching, burning, dryness, irritation and/or dyspareunia. In other women, atrophic vaginitis is asymptomatic. Atrophic vaginitis is associated with estrogen deficiencies and is accompanied by decreased numbers of lactobacilli, decreased lactic acid concentration, and/or increased vaginal pH.

Estrogen therapy (with naturally occurring or synthetic estrogenic compounds) can shift the composition of vaginal communities to become dominated by lactobacilli. However, there is a need for methods to shift the vaginal community composition while minimizing the risk of adverse side effects associated with estrogen administration.

### III. Manipulation of the Bacterial Composition of Vaginal Communities

The bacterial composition of vaginal communities, or microbiota, may be manipulated or altered by administering an estrogenic compound to a female subject. Increasing a level of estrogen, or estrogenic compound, in a female subject may increase the relative abundance of vaginal Lactobacillus in the vaginal microbiota. It is advantageous, however, to administer a minimum effective dose of an estrogenic compound to the female subject to manipulate the bacterial composition of the vaginal microbiota while lessening the risk of adverse side effects, such as the risk of blood clots in the legs and lungs, and increased risk of breast cancer with continuous therapy. The female subject may be a woman of reproductive age, a perimenopausal woman, or a postmenopausal woman. In some embodiments, the female subject is a woman of reproductive age.

Before beginning treatment, a vaginal sample is obtained from the female subject, wherein the vaginal sample comprises a vaginal microbiota. The vaginal sample may be obtained by any suitable method including, but not limited to, wiping, swabbing, or scraping the vaginal surface, or by other mechanical means. Optionally, a wetting agent, buffer, lubricant or other agent can be employed to facilitate recovery of the sample.

The vaginal sample is analyzed to determine the constituent species of the vaginal microbiota, and initial analysis results are obtained. The initial analysis results may comprise a relative abundance of Lactobacillus in the vaginal microbiota, an absolute abundance of Lactobacillus in the vaginal microbiota, a vaginal pH, a vaginal lactic acid concentration, or any combination thereof. The initial analysis results may further comprise a relative abundance of one or more particular Lactobacillus species in the vaginal sample, a total abundance of one or more particular Lactobacillus species in the vaginal sample, ratios of two or more particular Lactobacillus species in the vaginal sample, or any combination thereof. The particular Lactobacillus species may include L. crispatus, L. jensenii, L. gasseri, L. iners, L. coleohominis, L. johnsonii, or any combination thereof. Methods of determining the relative or absolute abundance of Lactobacillus in the vaginal microbiota and/or an absolute abundance of total bacteria in the vaginal microbiota are described in detail below. In some examples, the analysis is performed using a diagnostic test comprising using quantitative PCR, universal bacterial primers, and genus specific Lactobacillus primers to determine the relative abundance of Lactobacillus based on a ratio of Lactobacillus 16S rRNA gene copies to total bacterial 16S rRNA gene copies in the vaginal microbiota. The vaginal pH and/or vaginal lactic acid concentration may be determined by conventional methods known to those skilled in the art of biological sample analysis.

Based at least in part on the initial analysis results, an effective dose (or initial dose) of an estrogenic compound for the female subject is determined, and one or more effective doses of the estrogenic compound are administered to the female subject over an effective period of time. It is understood that “estrogenic compound” may refer to a single estrogenic compound or a combination of two or more different estrogenic compounds. An effective dose is a dose of the estrogenic compound that, when administered repeatedly (e.g., at periodic intervals) over the effective period of time, is sufficient to shift, or induce a change in, the relative abundance of vaginal Lactobacillus in the vaginal microbiota, the absolute abundance of vaginal Lactobacillus in the vaginal microbiota, the vaginal pH, the vaginal lactic acid concentration, or any combination thereof toward one or more determined target values. In certain embodiments, the initial effective dose may be sufficient to achieve one or more of the determined target values when administered periodically over the effective period of time. The effective period of time may range from a few days to several weeks or months. In some embodiments, the target value for the relative abundance of vaginal Lactobacillus or the absolute abundance of vaginal Lactobacillus is an abundance or concentration at which one or more Lactobacillus species are the predominant species in the vaginal microbiota. For example, the target relative abundance of vaginal Lactobacillus may be more than 50%. In certain embodiments, the target value for vaginal pH is a vaginal pH<5. The target value for lactic acid concentration may be a lactic acid concentration sufficient to provide a vaginal pH<5.

In some embodiments, determining the effective dose (or initial dose) of the estrogenic compound includes comparing one or more of (i) the relative abundance of Lactobacillus in the vaginal microbiota to a determined relative abundance of Lactobacillus target value, (ii) the absolute abundance of Lactobacillus in the vaginal microbiota to a determined absolute abundance of Lactobacillus target value, (iii) the vaginal pH to a determined vaginal pH target value, or (iv) the vaginal lactic acid concentration to a determined vaginal lactic acid concentration target value to provide a comparison, and selecting the effective dose of the estrogenic compound based at least in part on the comparison. For example, the effective dose of the estrogenic compound may be greater when the initial analysis results show a greater discrepancy from the desired target values. Other factors that may influence the effective dose include, but are not limited to, the female subject's age, weight, race, overall health and/or other health conditions, other medications taken by the female subject, the identity of the estrogenic compound, genetic polymorphisms in the female subject's estrogen receptors, the female subject's physiology (e.g., metabolism and excretion of estrogen), the female subject's diet (e.g., dietary content of phytoestrogens), and/or any combination thereof.

The effective dose may be titrated over a period of time to provide a female subject with a minimum effective dose to attain and/or maintain the determined target value(s) of the relative abundance of Lactobacillus in the vaginal microbiota, the total abundance of Lactobacillus in the vaginal microbiota, the vaginal pH, and/or vaginal lactic acid concentration. Thus, embodiments of the disclosed method include personalized dosing to the female subject. Titrating the effective dose includes obtaining sequential vaginal samples from a female subject over a period of time, analyzing each vaginal sample, and adjusting the effective dose for that female subject based at least in part on the analysis. Administering a minimum effective dose of the estrogenic compound to the female subject mitigates the risks of estrogenic therapy (e.g., increased risk of breast cancer, heart disease, heart attack, stroke, and/or blood clots).

Thus, in some embodiments, the method further includes obtaining a subsequent vaginal sample from the female subject a period of time after beginning administration of the one or more effective doses of the estrogenic compound, wherein the subsequent vaginal sample comprises a subsequent vaginal microbiota, which is typically different from the initial vaginal microbiota as a result of administering the estrogenic compound. The subsequent vaginal sample is analyzed and results of the analysis are obtained. The analysis results may include a relative abundance of Lactobacillus in the subsequent vaginal microbiota, an absolute abundance of Lactobacillus in the subsequent vaginal microbiota, a subsequent vaginal pH, a subsequent vaginal lactic acid concentration, or any combination thereof. Based at least in part on the subsequent analysis results, the effective dose of the estrogenic compound is adjusted to provide an adjusted effective dose (or subsequent dose) of the estrogenic compound for the female subject. Adjusting the effective dose of the estrogenic compound may include comparing one or more of (i) the relative abundance of Lactobacillus in the subsequent vaginal microbiota to the determined target value of the Lactobacillus relative abundance, (ii) the absolute abundance of Lactobacillus in the subsequent vaginal community to the determined target value of the Lactobacillus absolute abundance, (iii) the subsequent vaginal pH to the determined target value of the vaginal pH, or (iv) the subsequent vaginal lactic acid concentration to the determined target value of the vaginal lactic acid concentration to provide a comparison, and adjusting the effective dose based on the comparison. One or more adjusted effective doses (or subsequent doses) of the estrogenic compound is then administered to the female subject over an effective period of time, in response to which the one or more determined target values of the relative abundance of vaginal Lactobacillus in the vaginal microbiota, the absolute abundance of vaginal Lactobacillus in the vaginal microbiota, the vaginal pH, the vaginal lactic acid concentration, or any combination thereof is attained and/or maintained.

The effective dose of the estrogenic compound may be titrated over time by obtaining sequential vaginal samples from the female subject, analyzing the vaginal samples and adjusting the effective dose of the estrogenic compound periodically. Typically, vaginal samples may be obtained more frequently when beginning treatment with the estrogenic compound, and the frequency may be decreased over time. For example, vaginal samples may be obtained from the female subject once every week or once every two weeks for a period of 1-4 menstrual cycles, or 4-16 weeks, after administration of the estrogenic compound has begun. For example, samples may be obtained over a period of one more menstrual cycles, such as over a period of 1-4, 2-4, or 2-3 menstrual cycles, or a period of several weeks, such as a period of 4-16, 8-16, or 8-12 weeks. Frequent initial sampling allows the effective dose to be closely monitored and adjusted as needed to achieve and/or maintain the determined target value(s). After an initial period of weekly testing, or testing every two weeks, further vaginal samples may be obtained once every 3 to 12 months, such as once every 3 months, once every 6 months, or once every 12 months. Each sample is analyzed as described previously, and the effective dose of the estrogenic compound is adjusted as needed. Periodic vaginal sampling and analysis not only provides more precise dosing of the estrogenic compound, but also monitors the female subject to determine whether other factors (e.g., changes in overall health, weight, other medications, and the like) may affect the vaginal microbiota and warrant additional adjustments to the effective dose.

In certain embodiments, the effective dose is a minimum dose of the estrogenic compound effective to provide the female subject with a vaginal microbiota dominated by Lactobacillus species, and the method further includes administering the minimum dose of the estrogenic compound to the female subject, thereby producing in the female subject a vaginal microbiota dominated by Lactobacillus species. A person of ordinary skill in the art, such as a clinician with experience in the area of female reproductive health or experience in administering estrogenic compounds, will understand how to determine an effective dose, such as a minimum effective dose, of an estrogenic compound. The clinician may use information obtained from vaginal sample analysis, e.g., the microbial community composition, to aid in determining an effective dose.

In some embodiments, the effective dose of the estrogenic compound is a daily dose within a range of from 0.05 μg to 2 mg of the estrogenic compound. For example, the effective dose may be within a range of from 0.1 μg to 2 mg, 0.5 μg to 2 mg, 1 μg to 2 mg, 1 μg to 1.5 mg, 1 μg to 1 mg, 1 μg to 750 μg, 1 μg to 500 μg, lμg to 250 μg, 1 μg to 100 μg, lμg to 50 μg, 5 μg to 50 μg, or 5 μg to 35 μg.

The effective dose may be administered under any suitable dosing regimen. In one embodiment, the effective dose is administered daily. In another embodiment, the effective dose is administered at periodic intervals, such as weekly, every two weeks, or monthly. In an independent embodiment, an effective dose to be administered daily may be administered in divided doses over the course of a day; for example, the effective dose may be administered in two smaller doses at different times of day. The dosing regimen may include administering doses with overlapping therapeutic time windows. For example, the female subject may be administered a daily dose of an estrogenic compound having a therapeutic time window of more than one day. The effective dose may be administered by any suitable route. In some embodiments, the effective dose is administered orally, vaginally, or transdermally. The effective dose may be administered daily or at periodic intervals for a period of time sufficient to achieve a desired physiologic condition and/or the determined target value(s) of the relative abundance of vaginal Lactobacillus, the absolute abundance of vaginal Lactobacillus, the vaginal pH, the vaginal lactic acid concentration, or any combination thereof. The period of time may be at least one day, at least one week, at least one month, at least two months, at least three months, at least six months, at least one year, or longer. When administered as part of a PrEP regimen, administration may continue for the duration of the regimen. Alternatively, administration may continue while the female subject is sexually active, while the female subject is of reproductive age, or while the female subject is experiencing one or more vaginal symptoms of malodor, burning, itching, discharge, inflammation, or dyspareunia. Administration may continue for a period of time after the target relative abundance of vaginal Lactobacillus, the target absolute abundance of vaginal Lactobacillus, the target vaginal pH, the target vaginal lactic acid concentration, or any combination has been achieved. Administration also may continue for a period of time after completion of a PrEP regimen, after the female subject ceases to be sexually active, after the female subject is past reproductive age, or after any vaginal symptoms have been mitigated.

Suitable estrogenic compounds include, but are not limited to, estradiol, estrone, estriol, ethenyl estradiol, estrone sulfate, equilin, equilin sulfate, equilenin, estradiol 17 beta-cypionate, estradiol valerate, estradiol acetate, estradiol undecylate, polyestradiol phosphate, ethinylestradiol, methylestradiol, mestranol, moxestrol, quinestrol, benzestrol, dienestrol, dienestrol acetate, disethylstilbestrol dipropionate, fosfestrol, hexestrol, methestrol dipropionate, chlorotrianisene, doisynoestrol, methallenestril, 27-hydroxycholesterol, dehydroepiandrosterone (DHEA), 7-oxo-DHEA, 7α-hydroxy-DHEA, 16α-hydroxy-DHEA, 7β-hydroxepiandrosterone, 4-androstenedione, 5-androstenediol, 3α-androstanediol, a phytoestrogen, a mycoestrogen, and prodrugs thereof, solvates thereof, and all combinations thereof.

In some embodiments, administering the effective dose of the estrogenic compound comprises administering an amount of a pharmaceutical composition comprising the effective dose of the estrogenic compound. The pharmaceutical composition comprises at least one estrogenic compound and at least one additional component. For example, the pharmaceutical composition may include at least one estrogenic compound and a pharmaceutically acceptable carrier. The pharmaceutical composition may be provided in any suitable dosage form, such as an oral dosage form, a vaginal ring, a transdermal patch, or a topical cream, gel, ointment, paste, or spray comprising the pharmaceutical composition. In certain embodiments, a topical cream, ointment, gel, paste, or spray may be used since such dosage forms facilitate compounding pharmaceutical compositions comprising personalized effective doses of the estrogenic compound. Additionally, personalized dosing can be achieved by varying the amount of a topical cream, gel, ointment, paste, or spray applied, e.g., to vaginal tissue or skin.

Embodiments of the disclosed method are suitable for many uses, such as treatment of bacterial vaginosis, reducing the female subject's risk of acquiring a sexually-transmitted infection, increasing efficacy of a pre-exposure prophylaxis (PrEP) regimen, or treatment of other undesirable vaginal symptoms such as malodor, burning, itching, discharge, inflammation, dyspareunia, or any combination thereof.

A vaginal microbiota dominated by Lactobacillus species and/or an acidic vaginal pH may decrease a sexually active female subject's risk of acquiring a sexually transmitted infection (STI). Thus, in one embodiment, the method further includes determining that the female subject is sexually active, e.g., by obtaining a health and/or sexual activity history from the female subject. Administering the one or more effective doses of the estrogenic compound to the sexually active female subject reduces the female subject's risk of acquiring an STI compared to the risk of acquiring an STI in a female subject in the absence of estrogenic compound administration.

A vaginal microbiota dominated by Lactobacillus species and/or an acidic vaginal pH has been shown to increase efficacy of an anti-human immunodeficiency virus (anti-HIV) drug in a pre-exposure prophylaxis regimen (PrEP), thereby lessening a female subject's risk of acquiring HIV. Thus, in one embodiment, the method further includes determining that the female subject is a woman of reproductive age taking an antiviral drug in a PrEP regimen or selected to take an antiviral drug in a PrEP regimen. Administering the one or more effective doses of the estrogenic compound to the female subject increases efficacy of the PrEP regimen compared to an efficacy of a PrEP regimen for a female subject taking the antiviral drug in the absence of estrogenic compound administration. The antiviral drug may be an anti-HIV drug. Exemplary anti-HIV drugs administered in a PrEP regimen include, for example, tenofovir, emtricitabine, or a combination thereof.

A vaginal microbiota dominated by Lactobacillus species and/or an acidic vaginal pH has also been shown to decrease the incidence of undesirable vaginal symptoms such as malodor, burning, itching, discharge, inflammation, dyspareunia, or a combination thereof. Hence, in some embodiments, the method further includes determining that the female subject is experiencing one or more undesirable vaginal symptoms. Administering the one or more effective doses of the estrogenic compound to the female subject mitigates at least one of the one or more undesirable vaginal symptoms.

### IV. Identification of Predominant Species in Vaginal Samples

Constituent species in a vaginal sample are identified, providing a microbial profile that distinguishes the predominant species of microorganisms in the sample. A vaginal sample can be obtained by wiping, swabbing, or scraping the vaginal surface, or by other mechanical means. Optionally, a wetting agent, buffer, lubricant or other agent can be employed to facilitate recovery of the sample.

Once the sample is obtained, constituent species in the sample are determined. To prevent introduction of bias into the analysis, the constituent species of a sample advantageously are determined using a method that does not require preliminary culturing of the microorganisms. Identification of constituent species of microorganisms, including identification of the predominant species, establishes a microbial profile for the sample. Depending on the source of the sample, and on the status of the subject, for example, the health or disease status of the subject, the samples can include one or more predominant species of microorganisms. The species identified can include symbiotic microorganisms, commensal microorganisms and/or pathogenic microorganism. For example, in a sample obtained from a subject without a sign or symptom of a disease (e.g., a “normal” subject), the predominant species are likely to be symbiotic and/or commensal microorganisms. In contrast, pathogenic microorganisms are more likely to be observed in a sample from a subject with a disease, condition, symptom or sign related to a pathological condition. Thus, the methods described herein can be used to determine the communities of microorganisms present in both normal and disease (abnormal) states.

Culture-independent methods for identifying the constituent species in a sample of microorganisms involve detecting one or more molecular indicators of identity. A molecular indicator of identity can be any molecular species present in or produced by the microorganism, so long as it can be detected directly or indirectly. Preferably, the molecular species exists in sufficiently polymorphic forms that it can alone, or in combination with other molecular species, be used to determine the identity of the microorganism from which it is obtained.

Typically, the culture-independent methods involve preparing a nucleic acid sample from a sample of microorganisms, and detecting at least one molecular indicator of identity that can be used to determine the identity of the constituents of the sample. The nucleic acid can be DNA, RNA, or both, and can be prepared by any methods known in the art for the isolation and purification of nucleic acids. In some embodiments, the method includes polymerase chain reaction (PCR) amplification and sequencing of variable regions of bacterial 16S rRNA genes.

Amplification products can be produced using a variety of well-known protocols. PCR is an example of amplification. A biological sample is collected from a subject and contacted with oligonucleotide primers, under conditions that allow for the primers to hybridize to a nucleic acid template in the sample. The primers are extended under suitable conditions, dissociated from the template, and then re-annealed, extended, and dissociated to amplify the number of copies of the nucleic acid. Numerous procedures for PCR are known in the art. The product of amplification can be characterized by electrophoresis, restriction endonuclease cleavage patterns, oligonucleotide hybridization or ligation, and/or nucleic acid sequencing using standard techniques. Other examples of amplification include strand displacement amplification, as disclosed in U.S. Pat. No. 5,744,311; transcription-free isothermal amplification, as disclosed in U.S. Pat. No. 6,033,881; repair chain reaction amplification, as disclosed in WO 90/01069; ligase chain reaction amplification, as disclosed in EP-A-320 308; gap filling ligase chain reaction amplification, as disclosed in U.S. Pat. No. 5,427,930; and NASBA™ RNA transcription-free amplification, as disclosed in U.S. Pat. No. 6,025,134. Each of these patents and publications is incorporated herein by reference.

In some embodiments, the molecular indicator of identity can be detected by determining the nucleotide sequence of a portion of the microbial genome. Typically, the portion of the microbial genome includes one or more polymorphic polynucleotides, such as the 16S rRNA gene or any of the alternative phylogenetically informative genes discussed above. Methods for determining the nucleotide sequence of a nucleic acid are well established in the art. Additionally, numerous kits are available for manual and/or automated sequencing of nucleic acids.

One embodiment of the method involves preparing a nucleic acid sample including a molecular indicator of identity from at least one species of microbiota present in the vaginal sample and detecting the molecular indicator of identity. For example, the method can involve preparing at least one nucleic acid sample by preparing a DNA sample. As indicated above, the molecular indicator of identity can be a polymorphic polynucleotide, such as an rRNA gene (for example, a 16S rRNA gene). The molecular indicator of identity can be detected by determining the nucleotide sequence of the polymorphic polynucleotide, such as the 16S rRNA gene, or a portion or subsequence thereof. Alternative embodiments for detecting the molecular indicator of identity also include PCR with selective primers, quantitative PCR with selective primers, DNA-DNA hybridization, RNA-DNA hybridization, in situ hybridization, and combinations thereof. For example, the polymorphic polynucleotide can be detected by hybridization to a species-specific probe. In such an example, the species-specific probe hybridizes to a polymorphic target nucleic acid, such as a 16S rRNA gene. Optionally, the nucleic acid can be hybridized to at least one array comprising a plurality of species specific probes, e.g., a plurality of species specific probes, each of which identifies a species of vaginal microbiota. Detecting the molecular indicator of identity can also be accomplished using protein probes (such as antibodies) that bind to polymorphic target proteins, for example polymorphic target proteins that identify the species of vaginal microbiota.

### V. Pharmaceutical Compositions

Another aspect of the disclosure includes pharmaceutical compositions prepared for administration to a female subject and which include an effective dose of one or more estrogenic compounds. The effective dose of an estrogenic compound will depend on the identity of the estrogenic compound, the route of administration, and the physical characteristics of the subject being treated. Specific factors that may be taken into account include overall health, weight, age, race, diet, and concurrent medications.

Pharmaceutical compositions can include carriers, thickeners, diluents, buffers, preservatives, surface active agents and the like in addition to the one or more estrogenic compounds. Pharmaceutical compositions can also include one or more additional ingredients such as anti-inflammatory agents, anti-itching agents, topical anesthetics, and the like.

The estrogenic compounds disclosed herein can be administered to subjects by a variety of mucosal administration modes, including by oral delivery, transdermal delivery, or by topical delivery to other surfaces such as vaginal tissue or skin.

To formulate the pharmaceutical compositions, the estrogenic compounds can be combined with various pharmaceutically acceptable additives, as well as a base or vehicle for dispersion of the compound. Desired additives include, but are not limited to, pH control agents, such as arginine, sodium hydroxide, glycine, hydrochloric acid, citric acid, and the like. In addition, local anesthetics (for example, benzyl alcohol), isotonizing agents (for example, sodium chloride, mannitol, sorbitol), adsorption inhibitors (for example, Tween® 80 polyethylene sorbitol ester or Miglyol® 812 triglycerides), solubility enhancing agents (for example, cyclodextrins and derivatives thereof), stabilizers (for example, serum albumin), and reducing agents (for example, glutathione) can be included.

The estrogenic compounds can be dispersed in a base or vehicle, which can include a hydrophilic compound having a capacity to disperse the compound, and any desired additives. The base can be selected from a wide range of suitable compounds, including but not limited to, copolymers of polycarboxylic acids or salts thereof, carboxylic anhydrides (for example, maleic anhydride) with other monomers (for example, methyl (meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a base or vehicle, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters and the like can be employed as vehicles. Hydrophilic polymers and other vehicles can be used alone or in combination, and enhanced structural integrity can be imparted to the vehicle by partial crystallization, ionic bonding, cross-linking and the like. The vehicle can be provided in a variety of forms, including fluid or viscous solutions, gels, pastes, powders, microspheres and films for direct application to a mucosal surface.

The compositions of the disclosure can alternatively contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. For solid compositions, conventional nontoxic pharmaceutically acceptable vehicles can be used which include, for example, pharmaceutical grades of mannitol, lactose, starch, magnesium stearate, sodium saccharin, talcum, cellulose, glucose, sucrose, magnesium carbonate, and the like.

Pharmaceutical compositions for administering the estrogenic compounds can also be formulated as a cream, gel, ointment, paste, or spray. The vehicle can be a solvent or dispersion medium containing, for example, water, ethanol, polyol (for example, glycerol, propylene glycol, liquid polyethylene glycol, and the like), and suitable mixtures thereof. In many cases, it will be desirable to include isotonic agents, for example, sugars, polyalcohols, such as mannitol and sorbitol, or sodium chloride in the composition. Prolonged absorption of the compound can be brought about by including in the composition an agent which delays absorption, for example, monostearate salts and gelatin.

In certain embodiments, the estrogenic compounds can be administered in a time release formulation, for example in a composition which includes a slow release polymer. Time release formulations include, for example, transdermal patches and vaginal rings. These compositions can be prepared with vehicles that will protect against rapid release, for example a controlled release vehicle such as a polymer, microencapsulated delivery system or bioadhesive gel. Prolonged delivery in various compositions of the disclosure can be brought about by including in the composition agents that delay absorption, for example, aluminum monostearate hydrogels and gelatin. When controlled release formulations are desired, controlled release binders suitable for use in accordance with the disclosure include any biocompatible controlled release material which is inert to the active estrogenic compound(s) and which is capable of incorporating the compound and/or other biologically active agent. Numerous such materials are known in the art. Useful controlled-release binders are materials that are metabolized slowly under physiological conditions following their delivery (for example, at a mucosal surface, or in the presence of bodily fluids). Appropriate binders include, but are not limited to, biocompatible polymers and copolymers well known in the art for use in sustained release formulations. Such biocompatible compounds are non-toxic and inert to surrounding tissues, and do not trigger significant adverse side effects, such as nasal irritation, immune response, inflammation, or the like. They are metabolized into metabolic products that are also biocompatible and easily eliminated from the body.

Exemplary polymeric materials for use in the present disclosure include, but are not limited to, polymeric matrices derived from copolymeric and homopolymeric polyesters having hydrolyzable ester linkages. A number of these are known in the art to be biodegradable and to lead to degradation products having no or low toxicity. Exemplary polymers include polyglycolic acids and polylactic acids, poly(DL-lactic acid-co-glycolic acid), poly(D-lactic acid-co-glycolic acid), and poly(L-lactic acid-co-glycolic acid). Other useful biodegradable or bioerodable polymers include, but are not limited to, such polymers as poly(epsilon-caprolactone), poly(epsilon-caprolactone-CO-lactic acid), poly(epsilon.-caprolactone-CO-glycolic acid), poly(beta-hydroxy butyric acid), poly(alkyl-2-cyanoacrilate), hydrogels, such as poly(hydroxyethyl methacrylate), polyamides, poly(amino acids) (for example, L-leucine, glutamic acid, L-aspartic acid and the like), poly(ester urea), poly(2-hydroxyethyl DL-aspartamide), polyacetal polymers, polyorthoesters, polycarbonate, polymaleamides, polysaccharides, and copolymers thereof. Many methods for preparing such formulations are well known to those skilled in the art (see, for example, Sustained and Controlled Release Drug Delivery Systems, J. R. Robinson, ed., Marcel Dekker, Inc., New York, 1978).

In accordance with the various treatment methods of the disclosure, the estrogenic compound can be delivered to a subject in a manner consistent with conventional methodologies. In accordance with the disclosure herein, a prophylactically or therapeutically effective amount of the estrogenic compound(s) is administered to a subject in need of such treatment for a time and under conditions sufficient to prevent, inhibit, and/or ameliorate a selected disease or condition or one or more symptom(s) thereof.

The estrogenic compounds can be administered for either a prophylactic purpose, a therapeutic purpose, or both. When provided prophylactically, the estrogenic compound(s) is provided in advance of any symptom. The prophylactic administration of the estrogenic compound(s) serves to prevent or ameliorate any subsequent disease process. When provided therapeutically, the estrogenic compound(s) is provided at (or shortly after) the onset of a symptom of disease or infection.

For prophylactic and therapeutic purposes, the estrogenic compound(s) can be administered to the subject orally, via continuous delivery (for example, continuous transdermal or mucosal delivery) over an extended time period, or in a repeated administration protocol (for example, by a daily or weekly repeated administration protocol). The effective dosages of the estrogenic compound(s) can be provided as repeated doses within a prolonged prophylaxis or treatment regimen that will yield clinically significant results to alleviate one or more symptoms or detectable conditions associated with a targeted disease or condition as set forth herein. Determination of effective dosages in this context is typically guided by administration protocols that significantly reduce the occurrence or severity of targeted symptoms or conditions in the subject or that reduce the subject's risk of acquiring a sexually transmitted infection.

One commercially available pharmaceutical composition is Premarin® conjugated estrogens (Pfizer, Inc.), a pharmaceutical preparation containing a sodium-salt mixture of estrone sulfate (52-62%) and equilin sulfate (22-30%), with a total of the two between 80-88%. The potency of the preparation is expressed in terms of an equivalent quantity of sodium estrone sulfate. Another commercially available pharmaceutical composition is Estrace® vaginal cream (Allergan Pharmaceuticals), which contains estradiol. Commercially available vaginal rings include Estring® estradiol vaginal ring (Pfizer, Inc.) and Femring® estradiol acetate vaginal ring (Allergan Pharmaceuticals). There are many other available estrogenic compound-containing tablets, patches, creams, and other formulations. Alternatively, an estrogenic compound preparation may be specially compounded or formulated to suit the needs of a female subject.

### VI. Example

A vaginal sample is obtained from a female subject. The female subject may (i) be experiencing one or more undesirable vaginal symptoms, (ii) be sexually active, and/or (iii) be a woman of reproductive age taking an antiviral drug in a pre-exposure prophylaxis regimen or selected to take an antiviral drug in a PrEP regimen.

Initial analysis results of the vaginal sample are obtained, where the initial analysis results include a relative abundance of Lactobacillus in the vaginal microbiota, an absolute abundance of Lactobacillus in the vaginal microbiota, a vaginal pH, a vaginal lactic acid concentration, or any combination thereof. Based at least in part on the initial analysis results, a clinician determines an effective dose of an estrogenic compound for the female subject to attain a target relative abundance of vaginal Lactobacillus in the vaginal microbiota, a target absolute abundance of vaginal Lactobacillus in the vaginal microbiota, a target vaginal pH, a target vaginal lactic acid concentration, or any combination thereof.

The effective dose of the estrogenic compound is administered to the female subject. The effective dose may be administered daily, weekly, or at other period intervals for a time sufficient to achieve the target relative abundance of vaginal Lactobacillus in the vaginal microbiota, the target absolute abundance of vaginal Lactobacillus in the vaginal microbiota, the target vaginal pH, the target vaginal lactic acid concentration, or any combination thereof.

The female subject is monitored for a period of time to assess efficacy of the treatment and to titrate the effective dose to provide a personalized effective dose of the estrogenic compound. Monitoring is performed by obtaining one or more subsequent vaginal samples, obtaining analysis results of the subsequent vaginal sample(s), and adjusting the effective dose of the estrogenic compound based at least in part on the analysis results. The adjusted effective dose of the estrogenic compound then is administered to the female subject. The female subject may be monitored weekly or every two weeks for an initial period of time, such as for 1-4 menstrual cycles or 4-16 weeks. The female subject may then be monitored at less frequent intervals, such as once every 3-12 months, with the effective dose adjusted as needed to achieve and/or maintain a vaginal microbiota dominated by Lactobacillus species as evidenced by the target relative abundance of vaginal Lactobacillus in the vaginal microbiota, the target absolute abundance of vaginal Lactobacillus in the vaginal microbiota, the target vaginal pH, the target vaginal lactic acid concentration, or any combination thereof.

### Sample Obtention and Analysis

A vaginal sample and analysis results may be obtained by the following exemplary protocols (Shen et al., Sci Rep 2016, 6:24380; Yuan et al. PLoS ONE 2012, 11(9):e0163148). A person of ordinary skill in the art will understand, however, that other protocols may be used to obtain the vaginal sample and/or analysis results.

Sample Collection:

Vaginal swab samples are self-collected using the Copan ESwab™ (Copan Diagnostics Inc., USA) and then placed in a sterile tube that contains 1 mL of Amies transport medium. The swab samples are placed on ice immediately and transferred to a −80° C. freezer. All samples are kept frozen at −80° C. until they are analyzed. Just prior to analysis the samples are thawed on ice, then mixed by vortexing. 250 μL of each sample are transferred to bead beating tubes and 100 μL of a lytic enzyme cocktail are added that includes 50 μL lysozyme 500 kU/mL, 6 μL mutanolysin 25 kU/mL, 4 μL lysostaphin 3000 kU/mL, and 41 μL of 10 mM Tris-HCl and 50 mM EDTA pH 8.0. The samples are incubated at 37° C. for 1 h in a dry heat block. Next, 750 mg of zirconia-silica beads (0.1 mm mean diameter) are added to all samples and the tubes are placed in Mini-BeadBeater-96 (BioSpec Products, Inc., Bartlesville, Okla.) at room temperature for 1 min at 2100 rpm. Bead beating is followed by a brief centrifugation to settle the beads. Bacterial genomic DNA is isolated from supernatants using a QIAamp DNA Mini kit (Qiagen Inc., Valencia Calif.) according to the manufacturer's protocol. The amount of DNA in samples is quantified with a QuantiFluor dsDNA kit (Promega Inc., Madison Wis.) using a Turner TBS-380 mini-fluorimeter (Turner BioSystems, USA), while the size and integrity of the genomic DNA is verified using an Agilent DNA 1000 kit using an Agilent Bioanalyzer 2100 according to manufacturer's recommendations (Agilent Technologies Inc., USA).

Whole-Genomic DNA Extraction from Vaginal Swabs:

Vaginal swab specimens are thawed on ice and then vortexed for 5 minutes to suspend the cells. A 0.5 mL-aliquot is transferred to a sterile 2.0 mL tube with cell lysis buffer composed of 50 μL lysozyme (10 mg/mL, Sigma-Aldrich), 6 μL mutanolysin (25 KU/ml; Sigma-Aldrich, St. Louis, Mo., USA), and 3 μL lysostaphin (4000 U/ml, Sigma-Aldrich) and 41 μL of TE50 buffer (10 mM Tris-HCl and 50 mM EDTA, pH 8.0). After 1 hour of incubation at 37° C., 600 mg of 0.1-mm-diameter zirconia/silica beads (BioSpec, Bartlesville, Okla., USA) is added to the mixture and cells are mechanically disrupted using the Mini-BeadBeater-96 (BioSpec) at 2100 rpm for 1 minute. Further isolation and purification of the total genomic DNA from crude lysates are performed using QIAamp DNA Mini Kit (Qiagen, Hilden, GER) according to the manufacturer's recommendation except the DNA is eluted into two separate tubes using two 100 μL aliquots of AE buffer (10 mM Tris-HCl, 0.5 mM EDTA; pH 9.0). A PicoGreen® assay is used to quantify genomic DNA in each sample (Invitrogen, Carlsbad, Calif., USA). Fluorescence is determined using a Synergy™ HT Multi-Mode Microplate Reader (BioTek, Winooski, Vt., USA) at an excitation wavelength of 485 nm and emission wavelength of 528 nm.

PCR Amplification and Sequencing of the V1-V3 Region of Bacterial 16S rRNA Genes:

The variable V1-V3 regions of 16S rRNA genes in each sample are amplified in two rounds of PCR with dual barcode indexing prior to analysis on an Illumina MiSeq platform (Illumina, San Diego, Calif., USA). The first PCR round amplifies the target specific regions in 16S rRNA genes (E. coli positions 27F-534R), while the second attaches sample-specific barcodes and Illumina sequencing adapters. The PCR primer sequences are shown in Table 1.

Using the universal 16S rRNA primers 27F and 534R, the V1-V3 regions of 16S rRNA genes are amplified in 96-well microtiter plates using AmpliTaq Gold® DNA polymerase (Applied Biosystems) and 100 ng of template DNA in a total reaction volume of 50 μL. The first round of PCR is run in a PTC-100 thermal controller (MJ Research, St. Bruno, Quebec, CAN) using the following cycling parameters: 2 min of denaturation at 95° C., followed by 20 cycles of 1 min at 95° C. (denaturing), 1 min at 51° C. (annealing), and 1 min at 72° C. (elongation), with a final extension at 72° C. for 10 min. The presence of amplicons is confirmed by agarose gel electrophoresis and staining with SYBR® Green dye. The second PCR is run in a total reaction volume of 20 μL using the following parameters: 10 min of denaturation at 95° C., followed by 10 cycles of 15 s at 95° C. (denaturing), 30 s at 51° C. (annealing), and 1 min at 72° C. (elongation), with a final extension at 72° C. for 3 min. Negative controls without a template are included for each primer pair. The concentrations of amplicons are quantified by fluorometry (GeminiXPS, Molecular Devices, Sunnyvale, Calif., USA) using PicoGreen, then equimolar amounts (100 ng) of the PCR amplicons are pooled in a single tube. Short DNA fragments and amplification primers are removed from the pool amplicons using AMPure beads (Beckman-Coulter, Indianapolis, Ind., USA), and then the purified amplicons are recovered from a 1% agarose gel followed by a second size selection with AMPure beads. The resulting amplicon pool is amplified by PCR using Illumina adaptor specific primers and the PCR product is analyzed on a DNA 1000 chip for the Agilent 2100 Bioanalyzer (Agilent Technologies, Santa Clara, Calif., USA). When the entire purification procedure is completed, and no short fragments are observed after PCR, the final amplicon pool is then quantified using the KAPA Illumina® library quantification kit (KAPA Biosciences, Wilmington, Mass., USA) and the Applied Biosystems StepOnePlus™ real-time PCR system. The amplicons are sequenced using an Illumina MiSeq platform and a 250 bp paired-end protocol (Illumina, Inc., San Diego, Calif.) with custom sequencing primers (see Table 1) and 10% phiX DNA to increase sequence diversity.

Read Quality Control, Sequence Analysis, and Taxonomic Assignments:

Raw unclipped DNA sequence reads from the Illumina platform are cleaned, assigned and filtered in the following manner. Raw FASTQ files are analyzed for barcode assignment (Read 2 and 3, from the Illumina 4 read protocol) allowing for one mismatch. Amplicon primer sequences in Read 1 and 4 are identified using Cross Match (version 1.080806, parameters: min matches=8, min score=16) from the phred/phrap/consed application suite. Cross Match alignment information is then read into R and processed to identify alignment quality, directionality, barcode assignment, and read clip points. Base quality clipping is performed using the application Lucy (version 1.20p, parameters: max average error 0.002, max error at ends 0.002). The clipped reads are then aligned to the S LVA bacterial sequence database http://www.arb-silva.de using mothur (www.mothur.org/; version 1.27). Alignment end points are identified and used in subsequent filtering. Sequence reads are filtered to only those that meet the following criteria: (a) sequences are at least 100 bp in length; (b) max hamming distance of barcode=1; (c) maximum number of matching error to forward primer sequences=2; (d) have <2 ambiguous bases (Ns); (e) alignment to the SILVA bacterial database is within 75 bp of the expected alignment start and stop position; and (f) read alignment starts within the first 5 bp and extends through read to within the final 5 bp. The RDP Bayesian classifier is used to assign clipped and concatenated (Reads 1 and 4) sequences to phylotypes (RDP 2.5; http://rdp.cme.msu.edu). Reads are assigned to the first RDP level with a bootstrap score >=50. The proportions of various phylotypes in each sample are then calculated. The depth of coverage for each community is sufficient to detect taxa that constitute ≈0.1% of a community.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

TABLE 1
Barcoded PCR primers used for the amplification of 16S rRNA genes*.

| 27F Primer | Primer Sequence |
|---|---|
| 27F-YM1 | 5′-ACACTGACGACATGGTTCTACAGTAGAGTTTGATCCTGGCTCAG-3′ SEQ ID NO: 1 |
| 27F-YM2 | 5′-ACACTGACGACATGGTTCTACACGTAGAGTTTGATCCTGGCTCAG-3′ SEQ ID NO: 2 |
| 27F-YM3 | 5′-ACACTGACGACATGGTTCTACAACGTAGAGTTTGATCCTGGCTCAG-3′ SEQ ID NO: 3 |
| 27F-YM4 | 5′-ACACTGACGACATGGTTCTACATACGTAGAGTTTGATCCTGGCTCAG-3′ SEQ ID NO: 4 |
| 27F-Bif | 5′-ACACTGACGACATGGTTCTACAGTACGTAGAGTTTGATCCTGGCTCAG-3′ SEQ ID NO: 5 |
| 27F-Bor | 5′-ACACTGACGACATGGTTCTACACGTACGTAGAGTTTGATCCTGGCTCAG-3′ SEQ ID NO: 6 |
| 27F-Chl | 5′-ACACTGACGACATGGTTCTACAACGTACGTAGAGTTTGATCCTGGCTCAG-3′ SEQ ID NO: 7 |
| 534R Primer | Primer Sequence |
| 534R_1 | 5′-TACGGTAGCAGAGACTTGGTCTCCATTACCGCGGCTGCTGG-3′ SEQ ID NO: 8 |
| 534R2_ | 5′-TACGGTAGCAGAGACTTGGTCTGCCATTACCGCGGCTGCTGG-3′ SEQ ID NO: 9 |
| 534R_3 | 5′-TACGGTAGCAGAGACTTGGTCTTGCCATTACCGCGGCTGCTGG-3′ SEQ ID NO: 10 |
| 534R_4 | 5′-TACGGTAGCAGAGACTTGGTCTATGCCATTACCGCGGCTGCTGG-3′ SEQ ID NO: 11 |
| 534R_5 | 5′-TACGGTAGCAGAGACTTGGTCTCATGCCATTACCGCGGCTGCTGG-3′ SEQ ID NO: 12 |
| 534R_6 | 5′-TACGGTAGCAGAGACTTGGTCTTCATGCCATTACCGCGGCTGCTGG-3′ SEQ ID NO: 13 |
| 534R_7 | 5′-TACGGTAGCAGAGACTTGGTCTATCATGCCATTACCGCGGCTGCTGG-3′ SEQ ID NO: 14 |
| Adapter Primers | Primer Sequence |
| P5-CS1 | 5′-AATGATACGGCGACCACCGAGATCTACACNNNNNNNNACACTGACGACATGTTCTACA-3′ SEQ ID NO: 15 |
| P7-CS2 | 5′-CAAGCAGAAGACGGCATACGAGATNNNNNNNNTACGGTAGCAGAGACTTGGTCT-3′ SEQ ID NO: 16 |
| Sequencing Primers | Primer Sequence |
| FL1-CS1 | 5′-ACACTGACGACATGGTTCTACA-3′ SEQ ID NO: 17 |
| FL1-CS2 | 5′-TACGGTAGCAGAGACTTGGTCT-3′ SEQ ID NO: 18 |
| FL2-CS1rc | 5′-TGTAGAACCATGTCGTCAGTGT-3′ SEQ ID NO: 19 |
| FL2-CS2rc | 5′-AGACCAAGTCTCTGCTACCGTA-3′ SEQ ID NO: 20 |

*The underlined sequences are the universal 16S rRNA primers 27F and 534R, which includes seven different 27F primer sequences to capture a broad spectrum of taxa. The bold letters denote the universal sequence tags CS1 and CS2 included in both rounds of PCR primers and the italicized bases are added to the template specific primers to introduce variability of base calls during Illumina sequencing. The adapter primers include the Illumina specific sequence P5 as well as P7 for dual indexing, and the 8-bp barcode is denoted by eight italicized Ns which allow us to simultaneously sequence the amplicons from all samples using relatively few barcoded adapter primers and subsequenctly assign sequences to the corresponding samples from which they were obtained.

We claim:

1. A method, comprising:
obtaining a vaginal sample from a female subject, the vaginal sample comprising a vaginal microbiota;
obtaining initial analysis results of the vaginal sample, the initial analysis results comprising a relative abundance of Lactobacillus in the vaginal microbiota, an absolute abundance of Lactobacillus in the vaginal microbiota, a vaginal pH, a vaginal lactic acid concentration, or any combination thereof;
determining, based at least in part on the initial analysis results, an effective dose of an estrogenic compound for administration to the female subject to shift the relative abundance of vaginal Lactobacillus in the vaginal microbiota, the absolute abundance of vaginal Lactobacillus in the vaginal microbiota, the vaginal pH, the vaginal lactic acid concentration, or any combination thereof toward one or more determined target values; and
administering one or more effective doses of the estrogenic compound to the female subject over an effective period of time, in response to which the relative abundance of vaginal Lactobacillus in the vaginal microbiota, the absolute abundance of vaginal Lactobacillus in the vaginal microbiota, the vaginal pH, the vaginal lactic acid concentration, or any combination thereof shifts toward or attains the one or more determined target values.

2. The method of claim 1, where determining, based at least in part on the initial analysis results, the effective dose of the estrogenic compound for administration to the female subject further comprises:
comparing one or more of (i) the relative abundance of Lactobacillus in the vaginal microbiota to a determined relative abundance of Lactobacillus target value, (ii) the absolute abundance of Lactobacillus in the vaginal microbiota to a determined absolute abundance of Lactobacillus target value, (iii) the vaginal pH to a determined vaginal pH target value, or (iv) the vaginal lactic acid concentration to a determined vaginal lactic acid concentration target value to provide a comparison; and
selecting the effective dose of the estrogenic compound based at least in part on the comparison.

3. The method of claim 1, where the effective dose is administered daily or weekly to the female subject.

4. The method of claim 1, further comprising:
obtaining a subsequent vaginal sample from the female subject a period of time after beginning administration of the one or more effective doses of the estrogenic compound, the subsequent vaginal sample comprising a subsequent vaginal microbiota;
obtaining subsequent analysis results of the subsequent vaginal sample, the subsequent analysis results comprising a relative abundance of Lactobacillus in the subsequent vaginal microbiota, an absolute abundance of Lactobacillus in the subsequent vaginal microbiota, a subsequent vaginal pH, a subsequent vaginal lactic acid concentration, or any combination thereof;
adjusting, based at least in part on the subsequent analysis results, the effective dose of the estrogenic compound to provide an adjusted effective dose of the estrogenic compound for administration to the female subject; and
administering one or more adjusted effective doses of the estrogenic compound to the female subject over an effective period of time, in response to which the one or more determined target values of the relative abundance of vaginal Lactobacillus in the vaginal microbiota, the absolute abundance of vaginal Lactobacillus in the vaginal microbiota, the vaginal pH, the vaginal lactic acid concentration, or any combination thereof is attained and/or maintained.

5. The method of claim 4, where adjusting, based at least in part on the subsequent analysis results, the effective dose of the estrogenic compound further comprises:
comparing one or more of (i) the relative abundance of Lactobacillus in the subsequent vaginal microbiota to the determined target value of the Lactobacillus relative abundance, (ii) the absolute abundance of Lactobacillus in the subsequent vaginal community to the determined target value of the Lactobacillus absolute abundance, (iii) the subsequent vaginal pH to the determined target value of the vaginal pH, or (iv) the subsequent vaginal lactic acid concentration to the determined target value of the vaginal lactic acid concentration to provide a comparison;
adjusting the effective dose of the estrogenic compound based on the comparison.

6. The method of claim 4, further comprising titrating the effective dose of the estrogenic compound by performing the steps of obtaining a subsequent vaginal sample from the female subject, obtaining subsequent analysis results of the subsequent vaginal sample, and adjusting the effective dose of the estrogenic compound once every week or once every two weeks for a period of 4-16 weeks after beginning administration of the one or more effective doses of the estrogenic compound.

7. The method of claim 6, further comprising performing the steps of obtaining a subsequent vaginal sample from the female subject, obtaining subsequent analysis results of the subsequent vaginal sample, and adjusting the effective dose of the estrogenic compound once every 3-12 months after the period of 4-16 weeks.

8. The method of claim 1, where the effective dose is a minimum dose of the estrogenic compound effective to provide the female subject with a vaginal microbiota dominated by Lactobacillus species, the method further comprising administering the minimum dose of the estrogenic compound to the female subject, thereby providing the female subject with a vaginal microbiota dominated by Lactobacillus species.

9. The method of claim 1, where the effective dose is a daily dose within a range of from 0.05 μg to 2 mg of the estrogenic compound.

10. The method of claim 1, where obtaining analysis results of the vaginal sample comprises using a diagnostic test comprising using quantitative PCR, universal bacterial primers, and genus-specific Lactobacillus primers to determine the relative abundance of Lactobacillus based on a ratio of Lactobacillus 16S rRNA gene copies to total bacterial 16S rRNA gene copies in the vaginal microbiota.

11. The method of claim 1, where the effective dose of the estrogenic compound is administered orally, vaginally, or transdermally.

12. The method of claim 1, where the estrogenic compound comprises estradiol, estrone, estriol, ethinyl estradiol, estrone sulfate, equilin, equilin sulfate, equilenin, estradiol 17 beta-cypionate, estradiol valerate, estradiol acetate, estradiol undecylate, polyestradiol phosphate, ethinylestradiol, methylestradiol, mestranol, moxestrol, quinestrol, benzestrol, dienestrol, dienestrol acetate, disethylstilbestrol dipropionate, fosfestrol, hexestrol, methestrol dipropionate, chlorotrianisene, doisynoestrol, methallenestril, 27-hydroxycholesterol, dehydroepiandrosterone (DHEA), 7-oxo-DHEA, 7α-hydroxy-DHEA, 16α-hydroxy-DHEA, 7β-hydroxepiandrosterone, 4-androstenedione, 5-androstenediol, 3α-androstanediol, a phytoestrogen, a mycoestrogen, or any combination thereof.

13. The method of claim 1, where administering the effective dose of the estrogenic compound comprises administering an amount of a pharmaceutical composition comprising the effective dose of the estrogenic compound and a pharmaceutically acceptable carrier.

14. The method of claim 13, where the pharmaceutical composition is provided as an oral dosage form, a vaginal ring, a transdermal patch, or a topical cream, gel, ointment, paste, or spray comprising the pharmaceutical composition.

15. The method of claim 1, further comprising determining that the female subject is experiencing one or more vaginal symptoms of malodor, burning, itching, discharge, inflammation, or dyspareunia, and administering the one or more effective doses of the estrogenic compound to the female subject mitigates at least one of the one or more vaginal symptoms.

16. The method of claim 1, further comprising determining that the female subject is sexually active, and administering the one or more effective doses of the estrogenic compound to the female subject reduces the female subject's risk of acquiring a sexually transmitted infection (STI) compared to a risk of acquiring an STI in a female subject in the absence of estrogenic compound administration.

17. The method of claim 1, further comprising determining that the female subject is a woman of reproductive age taking an antiviral drug in a pre-exposure prophylaxis (PrEP) regimen or selected to take an antiviral drug in a PrEP regimen, and administering the one or more effective doses of the estrogenic compound to the female subject increases efficacy of the PrEP regimen compared to an efficacy of a PrEP regimen for a female subject taking the antiviral drug in the absence of estrogenic compound administration.

18. The method of claim 17, where the antiviral drug is an anti-human immunodeficiency virus (anti-HIV) drug.

19. The method of claim 18, where the anti-HIV drug is tenofovir, emtricitabine, or a combination thereof.

20. The method of claim 1, where the female subject is a woman of reproductive age.

21. The method of claim 1, where the initial analysis results further comprise a relative abundance of one or more particular Lactobacillus species in the vaginal sample, an absolute abundance of one or more particular Lactobacillus species in the vaginal sample, ratios of two or more particular Lactobacillus species in the vaginal sample, or any combination thereof.

22. The method of claim 21, where the particular Lactobacillus species comprise L. crispatus, L. jensenii, L. gasseri, L. iners, L. coleohominis, L. johnsonii, or any combination thereof.


SEQUENCE LISTING (20 sequences)

SEQ ID NO: 1 - DNA, 44 bp, Artificial Sequence
  Synthetic oligonucleotide
acactgacgacatggttctacagtagagtttgatcctggctcag

SEQ ID NO: 2 - DNA, 45 bp, Artificial Sequence
  Synthetic oligonucleotide
acactgacgacatggttctacacgtagagtttgatcctggctcag

SEQ ID NO: 3 - DNA, 46 bp, Artificial Sequence
  Synthetic oligonucleotide
acactgacgacatggttctacaacgtagagtttgatcctggctcag

SEQ ID NO: 4 - DNA, 47 bp, Artificial Sequence
  Synthetic oligonucleotide
acactgacgacatggttctacatacgtagagtttgatcctggctcag

SEQ ID NO: 5 - DNA, 48 bp, Artificial Sequence
  Synthetic oligonucleotide
acactgacgacatggttctacagtacgtagggtttgatcctggctcag

SEQ ID NO: 6 - DNA, 49 bp, Artificial Sequence
  Synthetic oligonucleotide
acactgacgacatggttctacacgtacgtagagtttgatcctggctcag

SEQ ID NO: 7 - DNA, 50 bp, Artificial Sequence
  Synthetic oligonucleotide
acactgacgacatggttctacaacgtacgtagaatttgatcttggttcag

SEQ ID NO: 8 - DNA, 41 bp, Artificial Sequence
  Synthetic oligonucleotide
tacggtagcagagacttggtctccattaccgcggctgctgg

SEQ ID NO: 9 - DNA, 42 bp, Artificial Sequence
  Synthetic oligonucleotide
tacggtagcagagacttggtctgccattaccgcggctgctgg

SEQ ID NO: 10 - DNA, 43 bp, Artificial Sequence
  Synthetic oligonucleotide
tacggtagcagagacttggtcttgccattaccgcggctgctgg

SEQ ID NO: 11 - DNA, 44 bp, Artificial Sequence
  Synthetic oligonucleotide
tacggtagcagagacttggtctatgccattaccgcggctgctgg

SEQ ID NO: 12 - DNA, 45 bp, Artificial Sequence
  Synthetic oligonucleotide
tacggtagcagagacttggtctcatgccattaccgcggctgctgg

SEQ ID NO: 13 - DNA, 46 bp, Artificial Sequence
  Synthetic oligonucleotide
tacggtagcagagacttggtcttcatgccattaccgcggctgctgg

SEQ ID NO: 14 - DNA, 47 bp, Artificial Sequence
  Synthetic oligonucleotide
tacggtagcagagacttggtctatcatgccattaccgcggctgctgg

SEQ ID NO: 15 - DNA, 59 bp, Artificial Sequence
  Synthetic oligonucleotide
  misc_feature (30)..(37): n is a, c, g, or t
aatgatacggcgaccaccgagatctacacnnnnnnnnacactgacgacatggttctaca

SEQ ID NO: 16 - DNA, 54 bp, Artificial Sequence
  Synthetic oligonucleotide
  misc_feature (25)..(32): n is a, c, g, or t
caagcagaagacggcatacgagatnnnnnnnntacggtagcagagacttggtct

SEQ ID NO: 17 - DNA, 22 bp, Artificial Sequence
  Synthetic oligonucleotide
acactgacgacatggttctaca

SEQ ID NO: 18 - DNA, 22 bp, Artificial Sequence
  Synthetic oligonucleotide
tacggtagcagagacttggtct

SEQ ID NO: 19 - DNA, 22 bp, Artificial Sequence
  Synthetic oligonucleotide
tgtagaaccatgtcgtcagtgt

SEQ ID NO: 20 - DNA, 22 bp, Artificial Sequence
  Synthetic oligonucleotide
agaccaagtctctgctaccgta